(19) United States
(12) Patent Application Publication
(10) Pub. No.: US 2010/0301655 A1
(43) Pub. Date: Dec. 2, 2010

(54) PORTABLE CENTERING HEAD CUSHION

(76) Inventor: Michelle Mezger, Dallas, TX (US)

Correspondence Address: OBER / KALER;C/O ROYAL W. CRAIG, 120 EAST BALTIMORE STREET, SUITE 800, BALTIMORE, MD, 21202, US

(21) Appl. No.: 12/788,670

(22) Filed: May 27, 2010

Related U.S. Application Data
(60) Provisional application No. 61/217,105, filed on May 27, 2009.

(51) Int. Cl. A47C 7/38; A47C 16/00; A61F 9/04

(52) U.S. Cl. 297/397; 5/655.4; 5/652; 2/15

(57) ABSTRACT

An assembly-free head cushion for toddlers traveling in a booster seat or regular auto seat, physically disabled persons, and for the general comfort of any vehicle passenger seeking a comfortable way to prop their head while seated on the couch, airplane, etc. The head cushion disclosed herein is attachable beneath the vehicle headrest to cushion and center passengers traveling in a vehicle. The comfort cushion is attached by a bean-filled ballast inserted beneath the headrest, and suspends downward to two opposing polyester-filler-stuffed pockets positioned on each side of the passenger to promote an erect position and generally provide cushioned support for passengers to ride in greater comfort over short and long distances, especially while sleeping.

### CROSS-REFERENCE TO RELATED APPLICATION(S)

The present application derives priority from U.S. provisional application No. 61/217,105 filed May 27, 2009.

### BACKGROUND OF THE INVENTION

1. Field of the Invention

The present invention relates to vehicle accessories and, more particularly, to a suspended head cushion for use when traveling as a passenger in a vehicle in order to maintain a comfortable upright position.

2. Description of the Background

Children are prone to falling asleep while traveling in a vehicle and often lack the requisite strength and ballast to maintain an erect head position, which causes their head to topple forward or sideward. This can become uncomfortable and even dangerous over a period of time. The same is often true for individuals who are physically injured or disabled, such as health patients, veterans and other with permanent or temporary disabilities. Indeed, when traveling over significant distances it can become uncomfortable for any passenger to maintain an erect head position.

There are many existing head cushions for comfort while traveling, but none address the foregoing problem.

For example, U.S. Pat. No. 6,793,287 to Dunk issued Sep. 21, 2004, teaches a supplemental head and body rest for use with a car seat. The head and body rest comprises a harness and two pillows that are suspended from the harness. The harness attaches to the vehicle's head rest, and the pillows are placed in the car seat to provide lateral restraint. The pillows are elongated members that run from the child's head to approximately mid-torso.

U.S. Pat. No. 6,758,526 to Marbutt issued Jul. 6, 2004, shows a head rest for a car seat. The head rest comprises two side cushions that fit around the infant's face and connect underneath the infant's chin. The cushions are secured to the car seat by laying a long flexible section of the base pad over the seat back.

U.S. Pat. No. 6,386,639 to McMichael issued May 14, 2002, discloses a head support that engages with a child seat. The head support includes a base sheet that has two side pillows that fit within a car seat. The pillows laterally support the head of a child. The base sheet wraps around the top of the car seat and is secured to the car seat by a strap assembly.

U.S. Pat. No. 5,056,533 to Solano issued Oct. 15, 1991, discloses a flexible support cushion that can be adapted for use with an automobile seat to be used as a booster. The support comprises a back and bottom portion. The back portion has a circumferential pad along its periphery for providing head and body support. The support apparatus is secured to the seat by using tie down straps and loops located along the periphery.

U.S. Pat. No. 4,862,536 to Pruit issued Sep. 5, 1989, discloses a back support device that fastens to a seat back. The device comprises a bonnet, a pair of spaced-apart flexible guide straps, and an inflatable support cushion. The bonnet fastens to the upper portion of a seat back. The guide straps are suspended from the bonnet, and a plurality of inflatable cushions are slidably mounted to the straps. Pruit disclose the use of a lumber support cushion having two elongated cylindrical portions along the cushion's periphery.

U.S. Pat. No. 4,738,488 to Camelio issued Apr. 19, 1988, shows an adjustable head rest that fastens to a chair back. A U-shaped pillow is connected to a fabric substrate, and an elongated fabric tab is connected to either the pillow or the substrate. The elongated tab fastens the head rest to the chair back.

U.S. Pat. No. 4,383,713 to Roston issued Mar. 17, 1983, shows an infant support apparatus comprising a planar sheet. Attached to the planar sheet are a pillow member for head support and two elongated lateral supports. Also attached to the planar sheet are two straps to secure the apparatus by draping the straps over the back rest.

Published U.S. Patent Application No. 2008/024541 by Mesika published Oct. 9, 2008, discloses affixing a weight to one end of a textile sheet. The weighted end is placed on a surface, and the other end is draped over the edge while being held in place.

Conventional infant car seats are design for infants traveling in the rear seat of a vehicle. There are no centering cushions for a front bucket seat.

What is needed is a portable protective cushion that may be readily positioned on a front bucket seat as well as rear seats, and which provides vertical support on each side of an individual (child as well as adult) who is sitting in the cushion to thereby promote an erect position and protect the user from injury in the event of vehicle side-to-side movement, and to generally provide cushioned support for any passenger to ride in greater comfort for short and long distances, especially while sleeping.

### SUMMARY OF THE INVENTION

It is, therefore, an object of the present invention to provide an assembly-free head cushion for individuals traveling in a vehicle seat, inclusive of toddlers traveling in a booster seat or regular auto seat, disabled persons, as well as any passenger seeking a comfortable way to prop their head while seated in a car, bus airplane, etc.

It is another object to provide a portable centering head cushion for passengers traveling in a vehicle which provides vertical support on each side of the head to promote an erect position and generally provide cushioned support for greater comfort when traveling over short and long distances, and while sleeping.

These and other objects are achieved herein by a suspended comfort cushion that suspends itself from the headrest of a vehicle seat to provide lateral head support, maintaining a passenger's head erect despite shifting of the vehicle. The comfort cushion comprises opposing fabric panels having an identical inverted T-shape, and sewn together and stitched to define a plurality of internal pockets. Two opposing vertical pockets are filled with polyester filler to provide the lateral support, and the upwardly extending tongue has opposing horizontal pockets filled with synthetic micro beads, beans or other weighted filler to provide suspension ballast and to help trap the comfort cushion beneath the headrest. To secure the comfort cushion to an automobile seat, the tongue is extended between the support posts of the headrest and is draped over the seat back, the headrest is clamped there atop, and the passenger simply sits back against it. The filled pockets of the extending tongue are spaced apart by approximately 0.5-1.5″ to leave an intermediate recess. When a passenger is seated atop the centering head cushion and desires to sleep, the tongue may be retrieved from behind the headrest and draped over passenger's head and eyes, the filled pockets tending to self-center over the eyes, thereby providing a sleep mask which applies a comforting pressure to the eyes.

### BRIEF DESCRIPTION OF THE DRAWINGS

Other objects, features, and advantages of the present invention will become more apparent from the following detailed description of the preferred embodiments and certain modifications thereof when taken together with the accompanying drawings in which:

FIG. 1 is a perspective front view of the comfort cushion 2 in use, according to an embodiment of the present invention.

FIG. 2 is a front view of the comfort cushion 2.

FIG. 3 is a side view of the comfort cushion 2 with tongue draped over the passenger's head and eyes to provide a sleep mask.

### DETAILED DESCRIPTION OF THE PREFERRED EMBODIMENT

The present invention is a suspending comfort cushion for a vehicle seat, including airline seats and automobile seats with or without extendable headrest. The comfort cushion self-suspends overtop the seat (under the headrest) by a weighted tongue to provide lateral support on opposing sides of the passenger, thereby centering the passenger. If the passenger wishes to sleep, the tongue is specially configured to be draped over the passenger's head and eyes to provide a sleep mask.

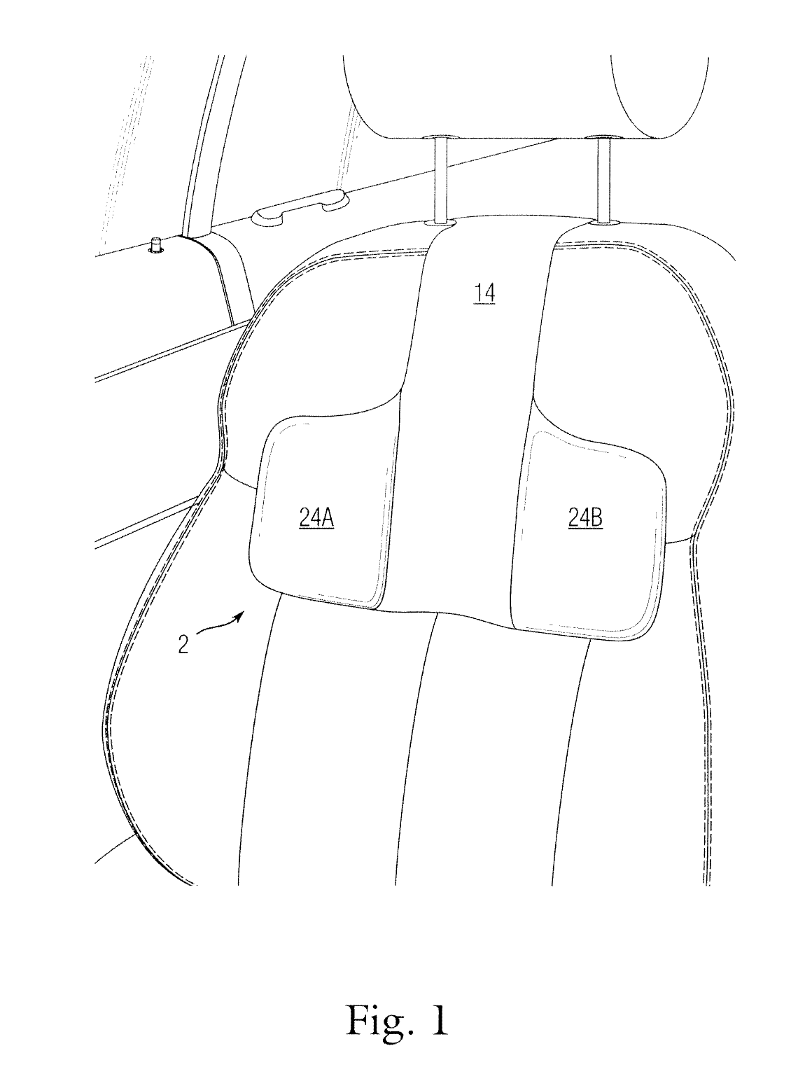

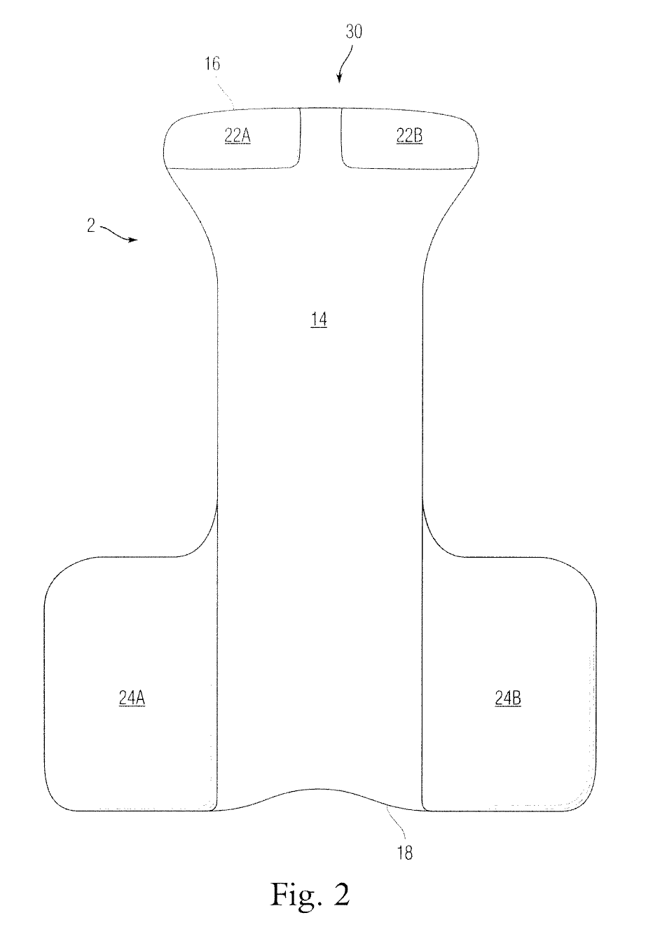

FIG. 1 is a perspective view of the comfort cushion 2 installed in a vehicle, and FIG. 2 is a full front view of the comfort cushion 2.

With combined reference to FIGS. 1 and 2, the comfort cushion 2 of the present invention may be manufactured from identical opposing fabric panels both having an inverted T-shape. The panels are sewn together around their entire periphery to define an enclosed bladder. One skilled in the art will readily understand that a single double-cut panel may be used if folded overtop onto itself and sewn shut. The inverted T-shape bladder defines an upwardly-extending tongue 14 which is preferably flared inward and outward as shown to its distal edge 16. The inverted T-shape bladder also defines broad lower section 17 approximately 2′ across and 1′ top-to-bottom, truncated at a lower distal edge 18. The entire peripheral seam including both upper and lower distally-sewn edges 16, 18 are preferably folded inward and sewn together using a standard box stitch.

In accordance with the present invention, the opposing panels 12 forming the bladder are sewn together medially to define a plurality of internal pockets. Specifically, with reference to FIG. 2 opposing upper pockets 22A, 22B are formed along the top of the tongue section 14 proximate the distal edge 16. In addition, two opposing vertical pockets 24A & 24B are formed along the distal sides of the lower lateral section 17. The upper pockets 22A, 22B formed along the top of the tongue section 14 are stuffed with synthetic micro beads (poly-fil micro beads made of polystyrene, organic beans or other weighted filled for ballast, while the two opposing lower lateral pockets 24A & 24B are filled with polyester filler for cushioning.

The two opposing lower lateral pockets 24A, 24B define spaced vertically-oriented pads suspending down from the headrest of the vehicle seat to provide lateral support, thereby tending to center the passenger and promote an erect position, generally providing cushioned support for passengers over short and long distances, and especially while sleeping.

The opposing lower lateral pockets 24A, 24B each comprise approximately a ⅔′ wide by 1′ tall cushioned pad, and they are spaced by a ⅔′-across flat tongue 14. The opposing lower lateral pockets 24A, 24B suspend down from the headrest of the vehicle seat to provide lateral support, thereby tending to center the passenger and promote an erect position as well as cushioned support for passengers.

As seen in FIG. 2, the bean-stuffed upper pockets 22A, 22B at the tip of upwardly extending tongue 14 are inserted under the headrest, and the weight of the beads/beans provides suspension ballast. In addition, the bulk of the beads/beans serve to trap the comfort cushion 2 beneath the seat headrest when the headrest is lowered down.

The bead-stuffed upper pockets 22A, 22B of the extending tongue are spaced apart by approximately 1.5″ to leave an intermediate recess 30. When a passenger is seated atop the centering comfort cushion 2 and desired to sleep, the tongue 14 may be retrieved from behind the seat and draped over passenger's head and eyes. The bead-filled pockets 22A, 22B center within the eye sockets and the recess 30 forms a nose bridge tending to keep the tongue 14 centered over the eyes, thereby providing a sleep mask which applies a comforting pressure to the eyes as the passenger sleeps.

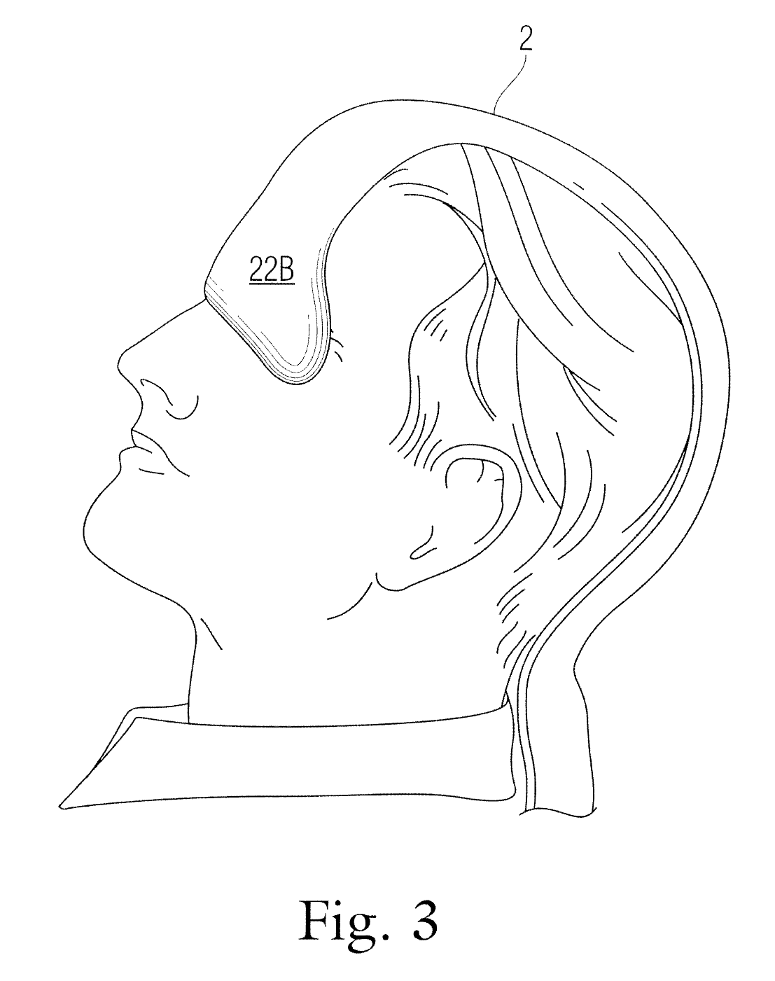

FIG. 3 is a side view of the comfort cushion 2 with tongue draped over the passenger's head and eyes to provide a sleep mask.

Indeed, it should be understood that various changes may be made in the form, details, arrangement and proportions of the parts. Such changes do not depart from the scope of the invention which comprises the matter shown and described herein and set forth in the appended claims.

I claim:

1. A comfort cushion, comprising:
a bladder having an inverted T-shape, said bladder having an upwardly-extending tongue extending to a broad lateral section, said bladder being sewn medially together to define a plurality of internal pockets, said pockets including a pair of upper pockets along a top edge of the tongue section for ballast, and two opposing vertical pockets formed along the distal sides of the lateral section, the pair of upper pockets being filled with weighted filler beads and the two opposing vertical pockets being filled with polyester filler.

2. The comfort cushion according to claim 1, wherein said weighted filler beads are organic beans.

3. The comfort cushion according to claim 1, wherein said weighted filler beads are polystyrene pellets.

4. The comfort cushion according to claim 1, wherein said pair of upper pockets are spaced apart to define a recessed nose bridge.

5. The comfort cushion according to claim 4, wherein said pair of upper pockets are spaced apart within a range of from 0.5-1.5″ to define a recessed nose bridge.

6. The comfort cushion according to claim 1, wherein said two opposing vertical pockets filled with polyester filler suspend down from the headrest of a vehicle seat to provide lateral support.

7. The comfort cushion according to claim 6, wherein when said two opposing vertical pockets are suspended down from the headrest of a vehicle seat to provide lateral support.

8. The comfort cushion according to claim 6, wherein when said opposing vertical pockets each comprise approximately a ⅔′ wide by 1′ tall pocket stuffed with said filler to define a cushioned pad.

9. The comfort cushion according to claim 8, wherein when said cushioned pads are spaced apart by a ⅔′-across flat tongue.

10. The comfort cushion according to claim 8, wherein when said cushioned pads are spaced apart by a ⅔′-across flat tongue.

11. The comfort cushion according to claim 1, wherein when said pair of upper pockets along a top edge of the tongue section are spaced apart so that said tongue section may be draped over a passenger's head and eyes to provide a sleep mask.

12. A comfort cushion, comprising:
a bladder having an inverted T-shape, said bladder having an upwardly-extending tongue extending to a broad lateral section, said bladder being sewn medially together to define a plurality of internal pockets, said pockets including a pair of upper pockets along a top edge of the tongue section for ballast, and a two opposing vertically-oriented pockets formed along the distal sides of the lateral section, the horizontal pocket being filled with weighted filler and the two vertical pockets stuffed being filled with polyester filler, said weighted upper pockets being spaced apart to define a recessed nose bridge, whereby said tongue may be selectively slung over a seat and suspended therefrom or draped over a passenger's head and eyes for use as a sleep mask.

13. The comfort cushion according to claim 12, wherein said pair of upper pockets are filled with pellets.

14. The comfort cushion according to claim 12, wherein said bladder is formed from two panels sewn together.

15. The comfort cushion according to claim 14, wherein said bladder is formed from two panels sewn together using an open box stitch.

16. The comfort cushion according to claim 13, wherein said pellets are organic beans.

17. The comfort cushion according to claim 13, wherein said pellets are polystyrene pellets.

18. The comfort cushion according to claim 13, wherein said pair of upper pockets are spaced apart to define a recessed nose bridge.

19. The comfort cushion according to claim 18, wherein said pair of upper pockets are spaced apart within a range of from 0.5-1.5″ to define a recessed nose bridge.

20. The comfort cushion according to claim 12, wherein said two opposing vertical pockets filled with polyester filler suspend down from the headrest of a vehicle seat to provide lateral support, and said opposing vertical pockets each comprise approximately a ⅔′ wide by 1′ tall pocket stuffed with said filler to define a cushioned pad, and said cushioned pads are spaced apart by a ⅔′-across flat tongue.
